(19) United States
(12) Patent Application Publication
(10) Pub. No.: US 2017/0007202 A1
(43) Pub. Date: Jan. 12, 2017

(54) SYSTEM AND METHOD OF HAPTIC FEEDBACK FOR TRANSESOPHAGEAL ECHOCARDIOGRAM ULTRASOUND TRANSDUCER PROBE

(71) Applicant: KONINKLIJKE PHILIPS N.V., Eindhoven (NL)

(72) Inventors: Michael Peszynski, Newburyport, MA (US); Carmen Goodwin, Woburn, MA (US); Daniel Thomas Nahill, Worcester, MA (US); Benjamin C. Sancetta, Worcester, MA (US); Bangyan Zhang, Worcester, MA (US)

(21) Appl. No.: 15/119,146

(22) PCT Filed: Mar. 2, 2015
(86) PCT No.: PCT/IB2015/051507

§ 371 Date: Aug. 16, 2016

Related U.S. Application Data
(60) Provisional application No. 61/951,717, filed on Mar. 12, 2014.

(51) Int. Cl. A61B 8/12; G01L 1/22; A61B 8/00; A61B 8/14; A61B 8/08

(52) U.S. Cl. CPC A61B 8/12; A61B 8/14; A61B 8/0883; A61B 8/4483; A61B 8/461; A61B 8/54; A61B 8/429; G01L 1/2262

(57) ABSTRACT

A system and method provide haptic feedback with respect to the maneuvering of a distal tip at a distal end of a transesophageal echocardiogram (TEE) ultrasound transducer probe deployed in a patient. An acoustic imaging system is connected to the TEE ultrasound transducer probe and produces acoustic images in response to one or more signals from the TEE ultrasound transducer probe. A control apparatus is provided for maneuvering the distal tip of the TEE ultrasound transducer probe with respect to the patient. A contact force sensing device senses a contact force between the distal tip of the TEE ultrasound transducer probe and the patient, and a feedback mechanism provides tactile, audio, and/or visual feedback when the contact force between the distal tip of the TEE ultrasound transducer probe and the patient exceeds a threshold force.

### TECHNICAL FIELD

This invention pertains to transesophageal echocardiogram (TEE) ultrasound transducer probes, and in particular a system and method of communicating haptic feedback to an operator of a TEE ultrasound transducer probe.

### BACKGROUND AND SUMMARY

Ultrasound systems are increasingly being employed in a variety of contexts. For example, ultrasound imaging is being increasingly employed in the context of minimally invasive surgeries,

In particular, a transesophageal echocardiogram (TEE) ultrasound transducer probe may be employed to provide live three dimensional cardiac imaging before or during a surgical procedure. Imaging capabilities are realized at the distal tip of the TEE ultrasound transducer probe, which contains components to produce ultrasonic waves and to detect ultrasonic echoes produced in response to the ultrasonic waves. The proximity of the gastrointestinal tract to the heart makes it an optimal conduit for ultrasound transmission. Once the TEE ultrasound transducer probe is positioned within the esophagus or stomach, an operator may employ manual controls to manipulate the depth of probe insertion and distal tip positioning to optimize the disposition of the TEE ultrasound transducer probe for imaging.

While operating the TEE ultrasound transducer probe, the operator may inadvertently maneuver or articulate the distal tip of the TEE ultrasound transducer probe beyond acceptable physical limits, causing potential esophageal or gastric trauma or injury to the patient. The manual controls may provide direct tactile feedback to the operator by stiffening or becoming harder to adjust as the distal tip approaches inherent articulation physical limits.

However, the present inventors have appreciated that the sensitivity of this crude form of haptic feedback may be inadequate in some situations to prevent the contact force of the distal tip of the TEE ultrasound transducer probe from exceeding safe limits to prevent esophageal or gastric trauma or injury to the patient. This particularly may be the case when and if robotically-controlled TEE ultrasound transducer probes are developed and deployed which lack tactile feedback to an operator.

Accordingly, it would be desirable to provide a system and method for supplying sensitive, robust, and responsive haptic feedback to an operator of a TEE ultrasound transducer probe, in particular to advise the operator in the event that the contact force between the distal tip of the TEE ultrasound transducer probe reaches or exceeds safe limits to prevent esophageal or gastric trauma or injury to the patient.

In one aspect of the invention, a system comprises: a transesophageal echocardiogram (TEE) ultrasound transducer probe having a distal tip at a distal end thereof; an acoustic imaging system connected to the TEE ultrasound transducer probe and configured to produce acoustic images in response to one or more signals from the TEE ultrasound transducer probe; a control apparatus for maneuvering the distal tip of the TEE ultrasound transducer probe with respect to a patient; a contact force sensing device for sensing a contact force between the distal tip of the TEE ultrasound transducer probe and the patient; and a feedback mechanism for providing at least one of tactile, audio and visual feedback when a contact force between the distal tip of the TEE ultrasound transducer probe and the patient exceeds a threshold force.

In some embodiments, the control apparatus comprises at least one control mechanism, and the feedback mechanism provides tactile feedback via the control mechanism.

In some embodiments, the tactile feedback includes increasing a resistance to further movement of the control mechanism in at least one direction when the contact force between the distal tip of the TEE ultrasound transducer probe and the patient exceeds the threshold force.

In some embodiments, the tactile feedback comprises inhibited articulation of the TEE ultrasound transducer probe in at least one direction via the control mechanism when the contact force between the distal tip of the TEE ultrasound transducer probe and the patient exceeds the threshold force.

In some embodiments, the feedback mechanism includes a user interface which provides at least one of: a visual indication via at least one light-producing element that the contact force exceeds the threshold force, and an audible sound indicating that the contact force exceeds the threshold force.

In some embodiments, the contact force sensing device comprises a plurality of force sensitive resistors disposed at the distal end of the TEE ultrasound transducer probe, wherein a resistance of at least one of the force sensitive resistors is a function of the contact force.

In some variations of these embodiments, the system further includes a processing device connected to at least one of the force sensitive resistors and which is configured to produce an output signal which is a function of the contact force.

In some versions of these embodiments, the processing device includes a Wheatstone bridge connected to at least one of the force sensitive resistors, and an amplifier connected to an output of the Wheatstone bridge.

In some embodiments, the control apparatus includes: at least one control mechanism; and at least one articulation cable connected between the control mechanism and the distal tip such that the control mechanism may maneuver the distal tip, wherein the contact force sensing device includes a torque sensor configured to sense a torque between the articulation cable and the control mechanism and in response thereto to produce an output signal which is a function of the contact force.

In some variations of these embodiments, the control mechanism comprises a gear apparatus, wherein the torque sensor includes at least one torque-measuring gear in the gear apparatus.

In some embodiments, the system includes a user interface which is configured to indicate a relationship between the contact force and the threshold force when the contact force does not exceed the threshold force.

In another aspect of the invention, a method comprises: maneuvering a distal tip at a distal end of a transesophageal echocardiogram (TEE) ultrasound transducer probe with respect to a patient; sensing a contact force between the distal tip of the TEE ultrasound transducer probe and the patient; and providing a feedback signal indicating when the contact force exceeds a threshold.

In some embodiments, the feedback signal causes an increased resistance to further movement in at least one direction by a control mechanism which is used to maneuver the distal tip of the TEE ultrasound transducer probe.

In some embodiments, the feedback signal triggers one of: an audible alert, and a visual alert when the contact force exceeds the threshold.

In some embodiments, the feedback signal further indicates a relationship between the contact force and the threshold force when the contact force does not exceed the threshold force.

In some embodiments, sensing the contact force between the distal tip of the TEE ultrasound transducer probe and the patient comprises sensing the contact force via a plurality of force sensitive resistors disposed at the distal end of the TEE ultrasound transducer probe.

In some versions of these embodiments, a processing device is connected to at least one of the force sensitive resistors and produces an output signal which is a function of the resistance of the force sensitive resistor, which in turn is a function of the contact force.

In some embodiments, sensing the contact force between the distal tip of the TEE ultrasound transducer probe and the patient comprises sensing a torque between a gear apparatus and at least one articulation cable which is connected between the gear apparatus and the distal tip such that the gear apparatus may maneuver the distal tip with respect to the patient.

In some versions of these embodiments, the torque is sensed by at least one torque-measuring gear in the gear apparatus.

In some embodiments, the method further includes inhibiting further articulation of the distal tip of the TEE ultrasound transducer probe which would cause the contact force to further exceed the threshold.

### BRIEF DESCRIPTION OF THE DRAWINGS

FIG. 1 shows one example embodiment of an acoustic imaging system with a transesophageal echocardiogram (TEE) ultrasound transducer probe.

FIG. 2 illustrates one example embodiment of a TEE probe and an associated control apparatus.

FIG. 3 illustrates four degrees of freedom for maneuvering the distal tip of a TEE ultrasound transducer probe.

FIG. 4 illustrates one example embodiment of a process of providing haptic feedback to an operator of a TEE ultrasound transducer probe to indicate when a contact force between the TEE ultrasound transducer probe and a patient exceeds a threshold.

FIG. 5 illustrates a portion of one example embodiment of a TEE ultrasound transducer probe having a plurality of force sensitive resistors disposed at the distal end thereof.

FIG. 6 plots contact force versus resistance for an example embodiment of a force sensitive resistor.

FIGS. 7A and 7B show portions of one embodiment of a processor for producing an output signal which is a function of a contact force sensed by a contact force sensor.

FIG. 8 illustrates one example embodiment of a torque-measuring gear mechanism for maneuvering a distal tip of a TEE ultrasound transducer probe.

FIG. 9 illustrates one example embodiment of a torque-measuring gear.

### DETAILED DESCRIPTION

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

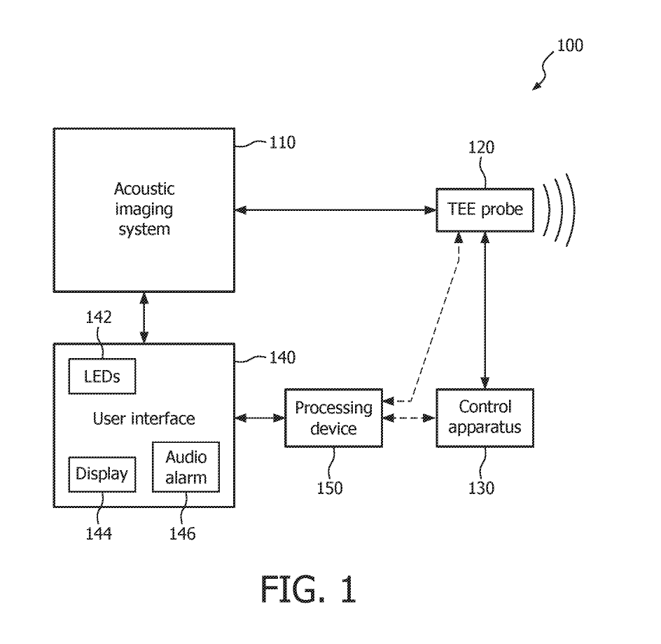

FIG. 1 shows one example embodiment of a system 100 including an acoustic imaging system 110 and an associated transesophageal echocardiogram (TEE) ultrasound transducer probe 120. Acoustic imaging system can be any suitable system for interfacing with TEE ultrasound transducer probe 120 to provide ultrasound imaging, for example three dimensional cardiac imaging which may be employed prior to or during surgery, for example minimally invasive surgery performed intravenously. TEE ultrasound transducer probe 120 includes an acoustic transducer, for example a one or two dimensional acoustic transducer array, disposed on a distal tip at a distal end thereof which may be positioned within the esophagus or stomach of a patient. TEE ultrasound transducer probe 120 may utilize electronically-steered microbeamforming technology to transmit the ultrasonic waves to an area of interest.

Via control apparatus 130, an operator may manually maneuver the distal tip to optimize the imaging produced via TEE ultrasound transducer probe 120.

In some embodiments, control apparatus 130 may comprise a handle having one or more control knobs which allow an operator to maneuver the distal tip of TEE ultrasound transducer probe 120 via one or more cables which pass through an insertion tube and are connected from the distal tip of TEE ultrasound transducer probe 120 to the control knobs. The cables may be maneuvered by turning the control knobs attached to the outside of the handle, in turn maneuvering the distal tip of TEE ultrasound transducer probe 120, In some embodiments, control apparatus 130 may allow for 4-way (2-plane) articulation of the distal tip and enhanced tip-to-patient contact.

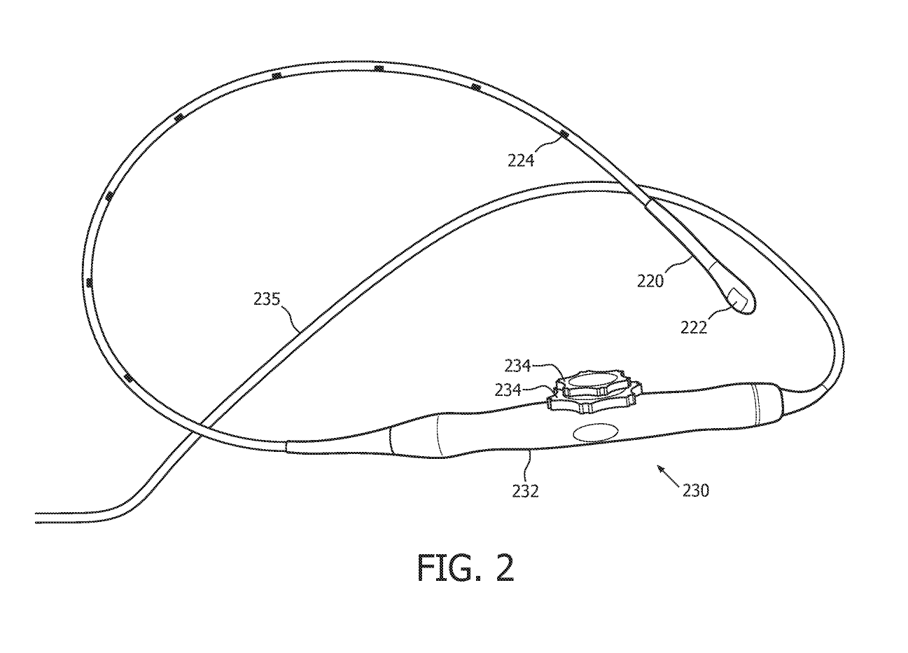

FIG. 2 illustrates one example embodiment of a TEE ultrasound transducer probe 220 and an associated control apparatus 230, which may respectively correspond to TEE probe 120 and control apparatus 130 in FIG. 1. TEE ultrasound transducer probe 220 has a distal tip 222 disposed at a distal end thereof, and is connected to control apparatus 230 at a proximal end thereof. Control apparatus 230 includes a probe handle 232 and one or more control mechanisms (e.g., knobs) 234. In some embodiments, within probe handle 232 a rack and pinion system may be attached to one articulation cables (also referred to as pull cables) disposed within the sheath of TEE ultrasound transducer probe 220 to drive articulation of the distal tip 222 thereof. Control knob(s) 234 may be associated with this rack and pinion system to thereby allow a user to maneuver or articulate distal tip 222 by manipulating the control knob(s) 234.

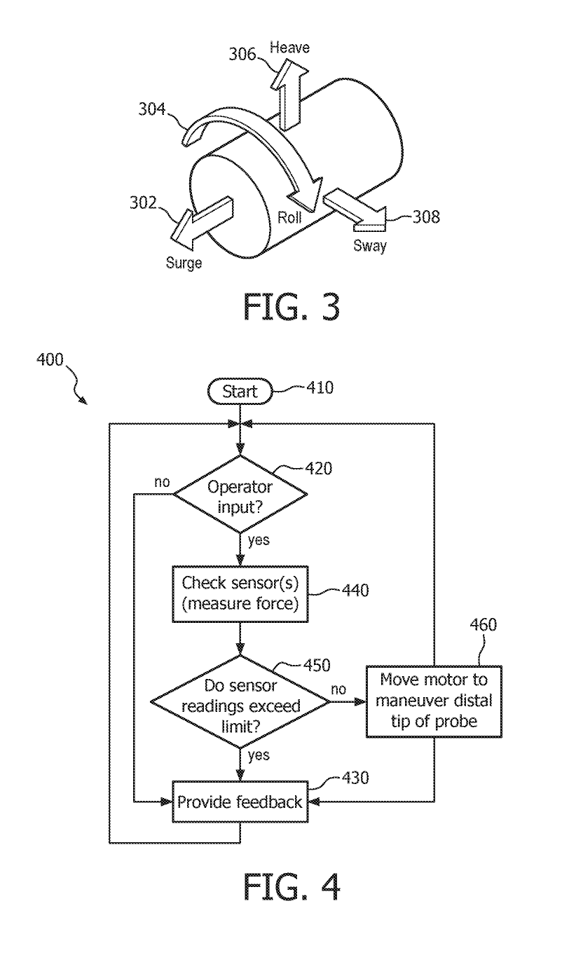

FIG. 3 illustrates four degrees of freedom—surge 302, roll 304, heave 306 and sway 308—for maneuvering the distal tip of TEE ultrasound transducer probe 120 which may be provided by control apparatus 230.

As noted above, it may happen in some cases that an operator may inadvertently maneuver the distal tip of TEE ultrasound transducer probe 120 beyond acceptable physical limits, causing potential esophageal or gastric trauma or injury to the patient.

Accordingly, system 100 provides to the operator haptic feedback with respect to the maneuvering of the distal tip at the distal end of TEE ultrasound transducer probe 120 deployed in a patient. Toward this end, system 100 includes a processing device 150 and a user interface 140, and at least one contact force responsive device or means which responds to the contact force between the distal tip of TEE ultrasound transducer probe 120 and a patient. In some embodiments, system 100 includes one or more contact force sensors deployed at the distal tip of TEE ultrasound transducer probe 120, and/or one or more contact force sensors associated with control apparatus 130, which may be deployed at the proximate end of TEE ultrasound transducer probe 120. Example embodiments of such a contact force responsive device or means will be described in greater detail below.

Dashed lines in FIG. 1 illustrate that in some embodiments, one or more contact force sensors which are provided at the distal tip of TEE ultrasound transducer probe 120 and are in communication with processing device 150, and in other embodiments, one or more contact force sensors which are provided associated with control apparatus 130, are in communication with processing device 150. In some embodiments, one or more contact force sensors may be provided both at the distal tip of TEE ultrasound transducer probe 120 and associated with control apparatus 130, and each of these contact force sensors are in communication with processing apparatus 150.

Processing device 150 produces an output signal which responds to the contact force between a patient and the distal tip of ultrasound transducer probe 120 from the one or more contact force sensors provided at the distal tip of TEE ultrasound transducer probe 120 and/or associated with control apparatus 130.

In some embodiments, processing device 150 may include a comparator which compares the output signal which responds to the contact force between a patient and the distal tip of TEE ultrasound transducer probe 120 to a threshold, and generates a feedback signal which indicates when the contact force between a patient and the distal tip of TEE ultrasound transducer probe 120 exceeds a threshold. In some embodiments, the threshold value may represent a maximum safe contact force that should be applied to a patient by the distal tip of TEE ultrasound transducer probe 120 to insure prevention of esophageal or gastric trauma or injury to the patient. In some embodiments, the threshold value may be determined during a calibration procedure for TEE ultrasound transducer probe 120 and/or system 100, which may be performed in situ.

In some embodiments, the feedback signal further may further indicate a relationship between the contact force and the threshold force when the contact force does not exceed the threshold force. For example, in some embodiments the feedback signal may indicate a relative level of the contact force when the contact force does not exceed the threshold force (e.g., a percentage of the threshold force), so that feedback may be provided to the operator to allow her/him to gauge or assess whether or not the threshold contact force is about to be reached.

In some embodiments, processing device 150 may include a microprocessor and memory, and additional circuitry such as analog-to-digital converters, etc. which may be employed to utilize the information obtained from the one or more contact force sensors of system 100 to produce the feedback signal. For example, in some embodiments one or more analog signals derived from the one or more contact force sensors of system 100 may be converted to digital signals and supplied to a microprocessor for comparison to one or more calibrated values for contact force levels—including for example a maximum threshold value—which may be stored in a memory. In some embodiments, such values may be obtained during a calibration procedure for system 100 and/or TEE ultrasound transducer probe 120.

In some embodiments, a feedback signal from processing device 150 may be provided to control apparatus 150 to inhibit or prevent further articulation of the distal tip of TEE ultrasound transducer probe 120 when the contact force between the distal tip and the patient exceeds a threshold. In that case, control apparatus 130 may provide tactile feedback to a user or operator which mimics the feedback a user may receive. For example, in some embodiments, a feedback signal from processing device 150 may be provided to cause a servo motor of control apparatus 130 to increase the resistance that the operator experiences to further movement of one or more control mechanisms in one or more directions when the contact force between the distal tip of the TEE ultrasound transducer probe and the patient exceeds the threshold force. In some embodiments, the feedback signal from processing device 150 may cause control apparatus 130 to inhibit or prevent further movement of one or more control mechanisms in one or more directions when the contact force between the distal tip of the TEE ultrasound transducer probe and the patient exceeds the threshold force.

In some embodiments, a feedback signal from processing device 150 may be supplied to user interface 140 to alert an operator when the contact force is deemed to be excessive and should be reduced by the operator. In some embodiments, user interface 140 may indicate a relative level of the contact force when the contact force does not exceed the threshold force, so that the operator may gauge or assess whether or not the threshold contact force is about to be reached.

In some embodiments, user interface 140 may include one or more lighting devices, such as an LED 142, which is illuminated or which blinks when the contact force exceeds the threshold to provide visible feedback to an operator of system 100. In some embodiments, the user interface may include a plurality of differently colored LEDs, wherein the color(s) of which of the LEDs are illuminated changes when the contact force exceeds the threshold (e.g., changes from illuminating one or more green LEDS to illuminating one or more red LEDs). In some embodiments user interface 140 may include a plurality of LEDs (e.g., five), wherein the number of LEDs which are illuminated corresponds to a relative contact force between TEE ultrasound transducer probe 120 and the patient. For example, when the contact force is low, then zero or one LED may be illuminated, while when the contact force exceeds the threshold, then all of the LEDs may be illuminated.

In some embodiments, user interface 140 may include a display device 144 such as an LCD display. In that case, in some embodiments a visual warning of excessive contact force may be provided on the LCD display, such as a warning text message (e.g., “Reduce Amount of Force Applied by Probe”), or an icon. Such a visual warning may be displayed using appropriate colors (e.g., in red) and/or may flash on the display to make them more noticeable to the operator.

In some embodiments, user interface 140 may further be incorporated into a display device for the acoustic images from acoustic imaging system 110. In that case, a visual warning of excessive contact force may be provided on the same display device which displays the acoustic images.

In some embodiments, user interface 140 may include a sound generating device 146, such as a bell, buzzer, or loudspeaker to provide audible feedback to an operator of system 100. In that case, an audible alert may be provided whenever the contact force between TEE ultrasound transducer probe 120 and the patient exceeds the threshold. In some embodiments, an audible feedback signal may be generated which indicates a relative contact force between TEE ultrasound transducer probe 120 and the patient. For example, the volume and/or pitch of an audible signal may be increased as the contact force increases. In some embodiments, one or more of the audible feedback devices and one or more of the visible feedback devices described above maybe used together, or may be user-selectable. A variety of other forms of feedback to an operator via user interface 140 are contemplated.

Any or all of the above forms of feedback may be provided in various embodiments of system 100 and TEE ultrasound transducer probe 120.

FIG. 4 illustrates one example embodiment of a process 400 of providing feedback to an operator when a contact force between TEE ultrasound transducer probe 120 and a patient exceeds a threshold.

The process starts at operation 410.

At operation 420 it is determined whether or not operator input is received to maneuver the distal tip of TEE ultrasound transducer probe 120.

If it is determined at operation 420 that no operator input is received, then the status quo is maintained and the current feedback which is being provided to the operator indicating the current contact force level remains unchanged and is continued to be provided to the operator at operation 430. It should be understood that the “current feedback” may include no active signaling, for example the current feedback may be that an alarm is turned off when the current contact force is less than the threshold. In that case, for example, a warning buzzer or LED may be turned off, etc.

If it is determined at operation 420 that operator input is received, then the one or more contact force sensors of system 100 are checked in operation 440.

In operation 450 it is determined from the contact sensors whether or not the operator's input will cause the contact force pressure to exceed a contact force threshold. As noted above, the threshold value may be determined during a calibration procedure for TEE ultrasound transducer probe 120 and/or system 100, which may be performed in situ.

If it is determined in operation 450 that the operator's input will cause the contact force pressure to exceed the threshold, then in operation 430 feedback is supplied to the user indicating that the threshold contact force is being exceeded, as described above. The process then returns to operation 420.

If it is determined in operation 450 that the operator's input will not cause the contact force pressure to exceed the threshold, then in operation 460 a motor or other motivating means is activated to maneuver the distal tip of TEE ultrasound transducer probe 120 as indicated by the user input in previous operation 420. The process then returns to operation 420.

Many variations of the process described above are of course possible.

Example embodiments of contact force sensors will now be described.

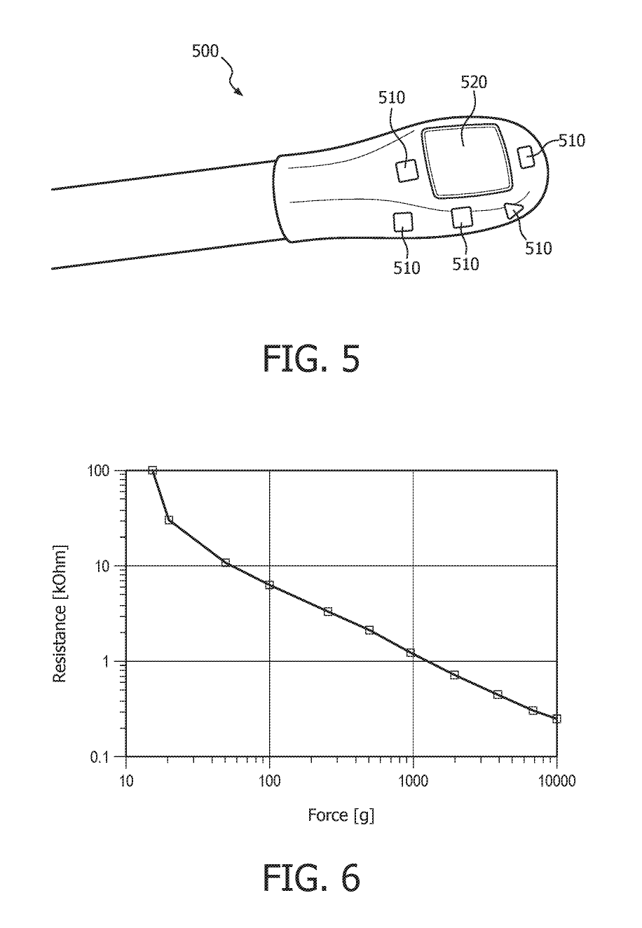

FIG. 5 illustrates a portion of one example embodiment of a TEE ultrasound transducer probe 500 having a plurality of force sensitive resistors 510 and an ultrasound transducer 520 disposed at the distal end thereof. TEE ultrasound transducer probe 500 maybe one embodiment of TEE ultrasound transducer probe 120 in system 300.

In TEE ultrasound transducer probe 500, force sensitive resistors 510 may directly measure the contact force between the distal tip of TEE ultrasound transducer probe 500 and the patient. Beneficially, force sensitive resistors 510 are disposed in locations at the distal tip which are considered most likely to experience patient contact. Although FIG. 5 shows an embodiment having four force sensitive resistors 510, in principle any number of force sensitive resistors 510 may be included. When more force sensitive resistors 510 are deployed, the likelihood of detecting any excessive contact force at any portion of the distal tip may be increased, however this may be at the expense of increased system complexity.

The resistance of each of the force sensitive resistors 510 changes in response to a deformation or strain to the pad as a result of the contact force which it experiences. In some embodiments, force sensitive resistors 510 may utilize a special design in order to change their electrical resistances based on the force exerted on them. This design may incorporate three separate layers sandwiched together with small gaps of air between. In that case, when force is applied to either of the two external layers, the external layers come into contact with the inner layer, decreasing the resistance. This decrease in resistance is then correlated to the force being exerted on force sensitive resistor 510. When there is no force applied, force sensitive resistor 510 may have almost infinite resistance

FIG. 6 plots contact force versus resistance for an example embodiment of a force sensitive resistor. It can be seen that in the example embodiment of FIG. 6, the resistance of force sensitive resistors 510 varies approximately linearly over a wide range of force values. In some embodiments, it may be advantageous to operate force sensitive resistors 510 over this linear range, or more specifically, to provide force sensitive resistors 510 where the threshold contact force value falls within this linear range of force sensitive resistors 510.

The resistance value of each force sensitive resistor 510 should be detected to determine whether or not the contact force between the distal tip of TEE ultrasound transducer probe 500 and the patient exceeds the threshold, and/or in some embodiments to determine the relative contact force between the distal tip of TEE ultrasound transducer probe 500 and the patient (for example, as a percentage of the threshold).

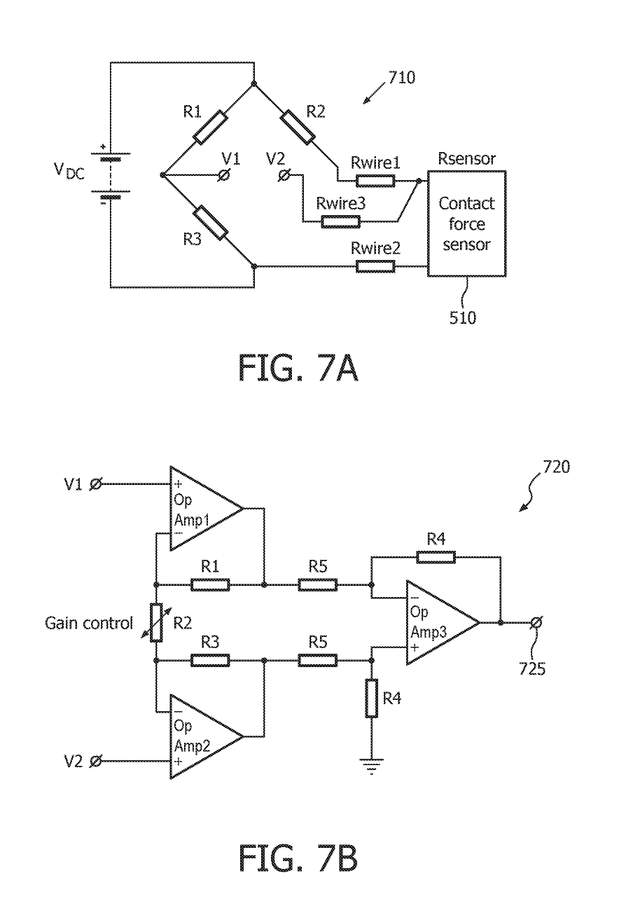

FIGS. 7A and 7B show portions of one embodiment of a processor for producing an output signal which is a function of a contact force sensed by a contact force sensor, for example force sensitive resistor 510.

FIG. 7A shows a Wheatstone bridge 710 which is used to produce a signal which is a function of the resistance of a contact force sensor, for example force sensitive resistor 510. In FIG. 7A, force sensitive resistor 510 is electrically connected to a DC voltage supply and energized within Wheatstone bridge 710. Upon deformation of the force sensitive resistor 510, resistive changes are detected within the circuitry. In the case where the electrical resistance of force sensitive resistor 510 varies linearly with contact force (see FIG. 6 above), the magnitude of the resultant signal from Wheatstone bridge 710 may be amplified and easily correlated to haptic force measurements. FIG. 7B illustrates one embodiment of such an amplifier 720.

In some embodiments, amplifier 720 produces an output signal 725 which may be compared to a threshold voltage corresponding to a maximum safe contact pressure for the distal tip TEE ultrasound transducer probe 500. The threshold voltage may be determined via a calibration procedure for TEE ultrasound transducer probe 220, 500 or system 100. Amplifier 720 includes gain control which may be adjusted to place the output signal within a proper range for comparison to the threshold voltage.

In some embodiments, output signal 725 may be compared to the threshold voltage via a comparator. In some embodiments, output signal 725 may be converted to a digital value via an analog-to-digital converter, and the digital value may be compared to a threshold voltage by a microprocessor. Other configurations are contemplated.

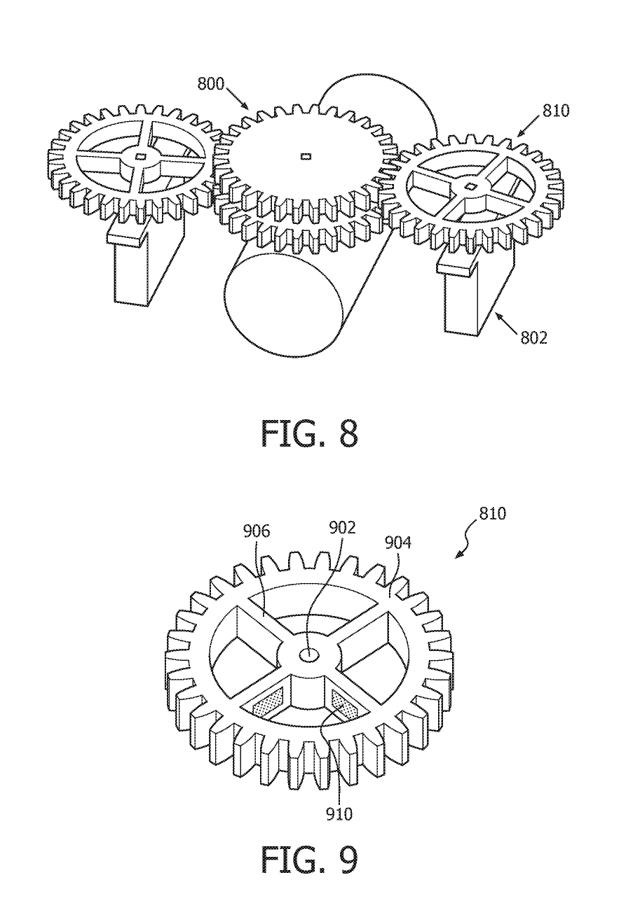

FIG. 8 illustrates one example embodiment of a torque-measuring gear mechanism 800 for maneuvering a distal tip of a TEE ultrasound transducer probe, such as TEE ultrasound transducer probe 120. In this arrangement, one or more servo motors 802 may be employed to operate associated torque-measuring gears 810. Torque-measuring gear mechanism 800 may be provided for a user or operator to employ as a control knob, or a more ergonomically friendly control knob structure may be provided on torque-measuring gear mechanism 800 to allow a user to operate the control mechanism.

FIG. 9 illustrates one example embodiment of a torque-measuring gear 810. Torque-measuring gear 810 includes an inner ring 902, an outer ring 904, and a plurality of spokes 906. A strain gauge 910 is provided on one or more of the spokes 906. One common type of strain gauge 910 consists of an insulating flexible backing which supports a metallic foil pattern. The gauge is attached to the object by a suitable adhesive. As torque-measuring gear 810 is deformed, the foil is deformed, causing its electrical resistance to change. This resistance change, may be measured using a Wheatstone bridge (see FIG. 6A), and is related to the strain by a quantity known as the gauge factor.

As noted above, in some embodiments, within the probe handle a rack and pinion system may be attached to pull or articulation cables to drive distal tip articulation. When approaching the physical limits of articulation, the compression sheath surrounding these cables within the bending neck may constrict greatly to cause pulling and friction between the cables. In turn, the control knobs on the handle may become increasingly more difficult to rotate. One or more spokes 906 of one or more torque-measuring gears 810 of torque-measuring gear mechanism 800 may experience strain translated from the handle pull cables. Strain gauges 910, strategically placed onto the torque-measuring gears 810, may be used to convert this mechanical deformation to an electrical signal as described above for feedback regarding the contact force between the distal tip of the TEE ultrasound transducer probe and the patient.

In some embodiments, frictional force generated by the articulation or pull cables may be captured, stored and characterized through an open loop feedback process during system use. This data may then be subtracted from the signal produced by strain gauges 910 to determine true haptic force measurements. Detection circuitry similar to that shown in FIGS. 7A and 7B may be employed with strain gauges 910 to generate a feedback signal for alerting an operator when the contact force between TEE ultrasound transducer probe 120 and a patient exceeds a patient.

While preferred embodiments are disclosed in detail herein, many variations are possible which remain within the concept and scope of the invention. Such variations would become clear to one of ordinary skill in the art after inspection of the specification, drawings and claims herein. The invention therefore is not to be restricted except within the scope of the appended claims.

1. A system, comprising:
a transesophageal echocardiogram (TEE) ultrasound transducer probe having a distal tip at a distal end thereof;
an acoustic imaging system connected to the TEE ultrasound transducer probe and configured to produce acoustic images in response to one or more signals from the TEE ultrasound transducer probe;
a control apparatus for maneuvering the distal tip of the TEE ultrasound transducer probe with respect to a patient;
a contact force sensing device for sensing a contact force between the distal tip of the TEE ultrasound transducer probe and the patient; and
a feedback mechanism for providing at least one of tactile, audio and visual feedback when a contact force between the distal tip of the TEE ultrasound transducer probe and the patient exceeds a threshold force,
wherein at least one of: the contact force sensing device comprises a plurality of force sensitive resistors disposed at the distal end of the TEE ultrasound transducer probe, wherein a resistance of at least one of the force sensitive resistors is a function of the contact force; and the control apparatus includes at least one control mechanism and at least one articulation cable connected between the control mechanism and the distal tip such that the control mechanism may maneuver the distal tip, wherein the contact force sensing device includes a torque sensor configured to sense a torque between the articulation cable and the control mechanism and in response thereto to produce an output signal which is a function of the contact force.

2. The system of claim 1, wherein the control apparatus comprises at least one control mechanism, and the feedback mechanism provides tactile feedback via the control mechanism.

3. The system of claim 1, wherein the tactile feedback includes increasing a resistance to further movement of the control mechanism in at least one direction when the contact force between the distal tip of the TEE ultrasound transducer probe and the patient exceeds the threshold force.

4. The system of claim 1, wherein the tactile feedback comprises inhibited articulation of the TEE ultrasound transducer probe in at least one direction via the control mechanism when the contact force between the distal tip of the TEE ultrasound transducer probe and the patient exceeds the threshold force.

5. The system of claim 1, wherein the feedback mechanism includes a user interface which provides at least one of: a visual indication via at least one light-producing element that the contact force exceeds the threshold force, and an audible sound indicating that the contact force exceeds the threshold force.

6. The system of claim 1, wherein the contact force sensing device includes the plurality of force sensitive resistors disposed at the distal end of the TEE ultrasound transducer probe.

7. The system of claim 4, further comprising a processing device connected to at least one of the force sensitive resistors and which is configured to produce an output signal which is a function of the contact force.

8. The system of claim 7, wherein the processing device includes:
a Wheatstone bridge connected to at least one of the force sensitive resistors; and
an amplifier connected to an output of the Wheatstone bridge to produce the output signal.

9. The system of claim 1, wherein the control apparatus includes:
the at least one control mechanism; and
the at least one articulation cable connected between the control mechanism and the distal tip such that the control mechanism may maneuver the distal tip,
wherein the contact force sensing device includes the torque sensor configured to sense the torque between the articulation cable and the control mechanism.

10. The system of claim 9, wherein the control mechanism comprises a gear apparatus, wherein the torque sensor includes at least one torque-measuring gear in the gear apparatus.

11. The system of claim 1, wherein, the system includes a user interface which is configured to indicate a relationship between the contact force and the threshold force when the contact force does not exceed the threshold force.

12. A method, comprising:
maneuvering a distal tip at a distal end of a transesophageal echocardiogram (TEE) ultrasound transducer probe with respect to a patient;
sensing a contact force between the distal tip of the TEE ultrasound transducer probe and the patient; and
providing a feedback signal indicating when the contact force exceeds a threshold.

13. The method of claim 12, wherein the feedback signal causes an increased resistance to further movement in at least one direction by a control mechanism which is used to maneuver the distal tip of the TEE ultrasound transducer probe.

14. The method of claim 12, wherein the feedback signal triggers one of an audible alert and a visual alert when the contact force exceeds the threshold.

15. The method of claim 12, wherein the feedback signal further indicates a relationship between the contact force and the threshold force when the contact force does not exceed the threshold force.

16. The method of claim 12, wherein sensing the contact force between the distal tip of the TEE ultrasound transducer probe and the patient comprises sensing the contact force via the plurality of force sensitive resistors disposed at the distal end of the TEE ultrasound transducer probe.

17. The method of claim 16, wherein a processing device is connected to at least one of the force sensitive resistors and produces an output signal which is a function of a resistance of the force sensitive resistor, which in turn is a function of the contact force.

18. The method of claim 12, wherein sensing the contact force between the distal tip of the TEE ultrasound transducer probe and the patient comprises sensing the torque between the gear apparatus and the at least one articulation cable which is connected between the gear apparatus and the distal tip such that the gear apparatus may maneuver the distal tip.

19. The method of claim 18, wherein the torque is sensed by at least one torque-measuring gear in the gear apparatus.

20. The method of claim 12, further comprising inhibiting further articulation of the distal tip of the TEE ultrasound transducer probe which would cause the contact force to further exceed the threshold.
